(19) United States
(12) Patent Application Publication
(10) Pub. No.: US 2007/0015828 A1
(43) Pub. Date: Jan. 18, 2007

(54) Highly selective serotonin and norepinephrine dual reuptake inhibitor and use thereof

(75) Inventors: Syed M. Shah, East Hanover, NJ (US); Eric C. Ehrnsperger, Chestnut Ridge, NY (US); Richard W. Saunders, Palisades, NY (US); Mahdi B. Fawzi, Morristown, NJ (US); Rocco J. Galante, Oakland, NJ (US); Garth T. Whiteside, West Windsor, NJ (US)

Correspondence Address: HOWSON AND HOWSON;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 11/485,663

(22) Filed: Jul. 13, 2006

Related U.S. Application Data
(60) Provisional application No. 60/699,665, filed on Jul. 15, 2005.

(51) Int. Cl. A61K 31/277; A61K 31/137

(52) U.S. Cl. 514/522; 514/649; 558/418; 564/338

(57) ABSTRACT

Highly selective dual serotonin and norepinephrine reuptake inhibitors are provided. These compounds have a lower side-effect profile and are useful in compositions and products for use in treatment of a variety of conditions including depression, fibromyalgia, anxiety, panic disorder, agorophobia, post traumatic stress disorder, premenstrual dysphoric disorder, attention deficit disorder, obsessive compulsive disorder, social anxiety disorder, generalized anxiety disorder, autism, schizophrenia, obesity, anorexia nervosa, bulimia nervosa, Gilles de la Tourette Syndrome, vasomotor flushing, cocaine and alcohol addiction, sexual dysfunction, borderline personality disorder, fibromyalgia syndrome, diabetic neuropathic pain, chronic fatigue syndrome, pain, visceral pain, Shy Drager syndrome, Raynaud's syndrome, Parkinson's Disease, and epilepsy.

### CROSS-REFERENCE TO RELATED APPLICATION

This application claims the benefit pursuant to 35 USC 119(e) of U.S. Provisional Patent Application No. 60/699,665, filed Jul. 15, 2005.

### BACKGROUND OF THE INVENTION

The market for neuroscience and women's health drugs has been moving towards the use of dual serotonin and norepinephrine reuptake inhibitors (SNRI) for first line treatment of various indications, as evidenced by the recent development of SNRI's such as Venlafaxine and Duloxetine. This contrasts with the traditional use of selective serotonin reuptake inhibitors (SSRI). Although the side-effect profile of SSRI's and SNRI's are less severe as compared to older, tricyclic antidepressants compounds, there are still some undesirable side effects related to the selectivity or other neuronal receptor binding (muscarinic, histamine and alpha-adrenergic, etc.) of these SSNI's and SNRI's. Binding to these receptors can lead to side effects such as, dry mouth, drowsiness, appetitite stimulation and some cardiovascular risks.

The higher norepinephrine (NE) activity of SNRI's has also been implicated in a number of side effects and therefore limits their application. For example, the currently available SNRI's have limited application for the treatment of irritable bowel syndrome (IBS) because of the constipation side effect associated with higher NE activity. Another potential side effect of SNRI's is that at higher dosages there is a modest increase in diastolic blood pressure and this side effect is associated with higher NE activity. Further, potential overdose situations have been associated with excess adrenergic stimulation, seizures, arrhythmias, bradycardia, hypertension, hypotension and death.

What are needed are alternative compositions for treating conditions associated with serotonin and norepinephrine imbalances, by allowing serotonin and or norepinephrine re-uptake inhibition for efficacy with lower post synaptic receptor binding for reduced side-effects [(H. Hall, et al., Acta pharmacol et. toxicol. 1984, 54, 379-384)].

### SUMMARY OF THE INVENTION

The present invention provides a new class of compounds with dual serotonin and norepinephrine reuptake inhibitor activity. Without wishing to be bound by theory, it is believed that these compounds will exhibit the reduced side effects due to binding with post synaptic neuronal receptors, for example histamine, muscarinic, alpha-adrenergic, serotonin (various types), dopamine, opiate, benzodiazepine, etc. This class of compounds is a more selective dual-reuptake inhibitor that has a different ratio of serotonin/norepinephrine reuptake inhibition activity than previous SNRI's.

In one aspect, the invention provides a compound of the structure:

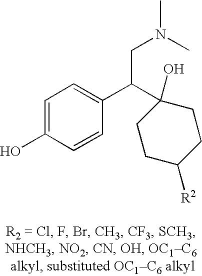

or a prodrug or a pharmaceutically acceptable salt thereof.

In another aspect, the invention provides a pharmaceutical composition comprising a compound of the invention and pharmaceutically acceptable carrier.

In still another aspect, the invention provides a method of using the compound of the invention for treating major depressive disorder, vasomotor symptoms, irritable bowel syndrome, premature ejaculation, pain and urinary incontinence in a subject in need thereof.

In a further embodiment, the invention provides methods of preparing compounds of formula A: 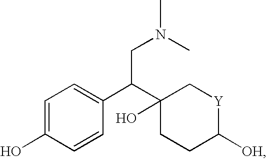
where Y is C or a bond.
or formula B: 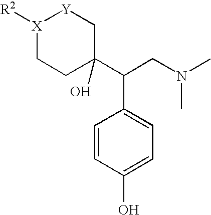
wherein X is C, N, or O; and Y is a C or absent; when X is C; R² is selected from H, halogen, CF₃, phenyl, SCH₃, OH, NHCH₃, OC₁-C₆ alkyl, and substituted OC₁-C₆alkyl; and when X is N, R² is selected from H, phenyl or CF₃.

These methods, described herein selectively provide compounds in the cis-configuration. In one embodiment, the compound of the invention is in a configuration is greater than 50% cis diastereomer. In another embodiment, the compounds of the invention are in a configuration which is greater than 95% cis diastereomer.

Still other aspects and advantages of the invention will be apparent from the following detailed description.

### BRIEF DESCRIPTION OF THE DRAWINGS

FIG. 1 provides an X-ray powder diffraction of 1-[-2-dimethylamino-1-(4-phenol)-ethyl-cis-1,4-cyclohexandiol.

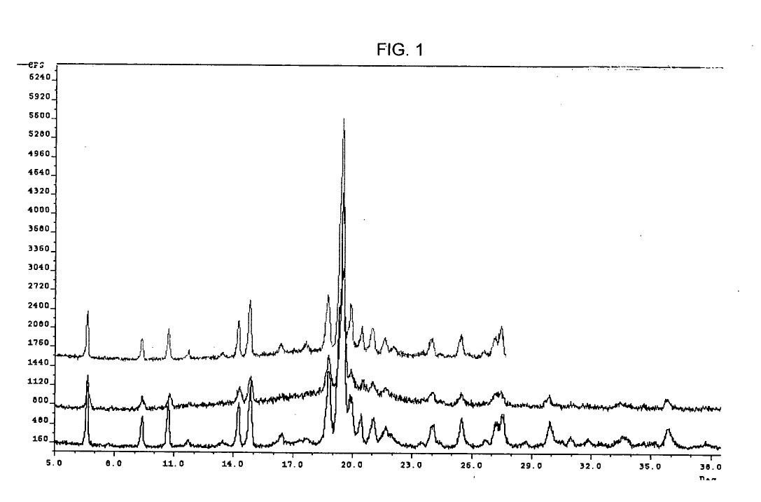

FIG. 2 provides a chart of the hygroscopicity profile of 1-[-2-dimethylamino-1-(4-phenol)-ethyl-cis-1,4-cyclohexandiol.

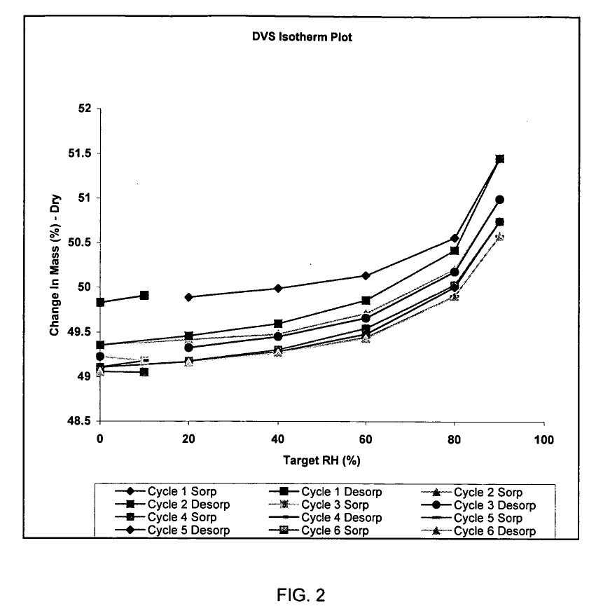

FIG. 3 provides a chart of the DSC of 1-[-2-dimethylamino-1-(4-phenol)-ethyl-cis-1,4-cyclohexandiol.

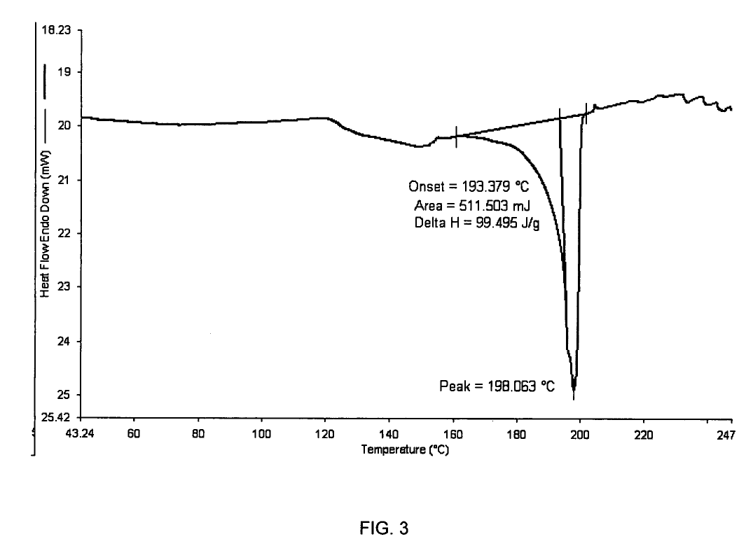

FIG. 4 provides a chart of the pH—solubility profile of 1-[-2-dimethylamino-1-(4-phenol)-ethyl-cis-1,4-cyclohexandiol.

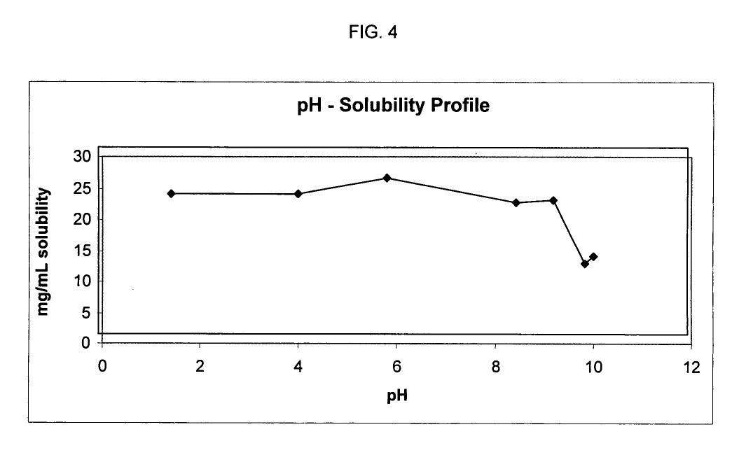

FIG. 5 provides an X-ray powder diffraction of 4-[2-dimethylamino-1-(cis-1-hydroxy-4-methoxy-cyclohexyl)-ethyl]-phenol.

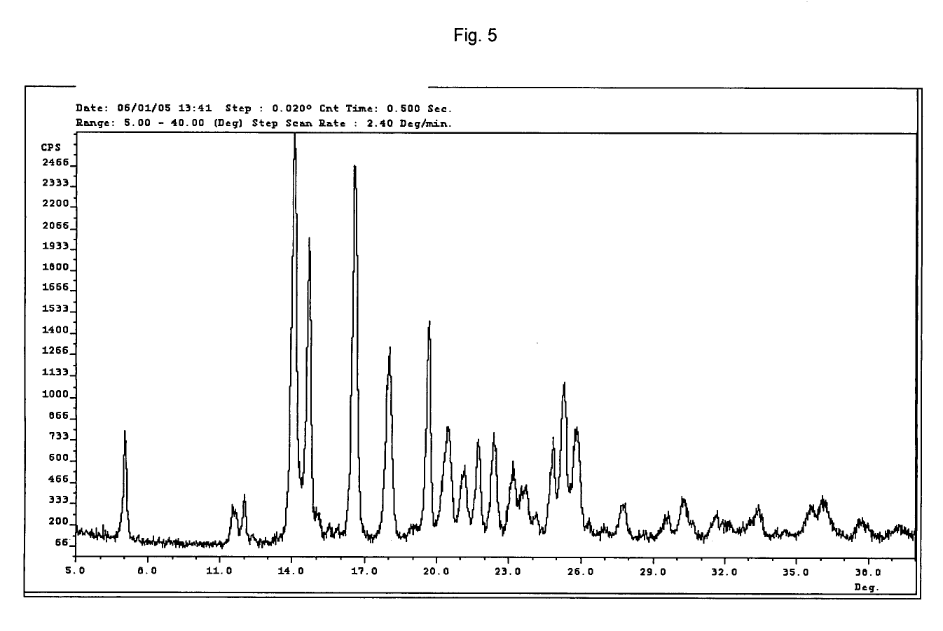

FIG. 6 provides a chart of the hygroscopicity profile of 4-[2-dimethylamino-1-(cis-1-hydroxy-4-methoxy-cyclohexyl)-ethyl]-phenol.

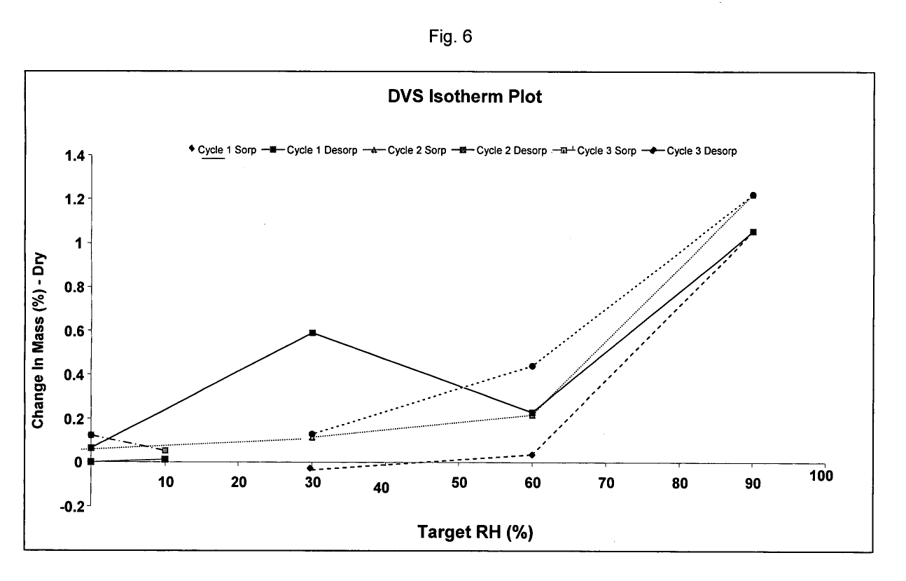

FIG. 7 provides a chart of the DSC of 4-[2-dimethylamino-1-(cis-1-hydroxy-4-methoxy-cyclohexyl)-ethyl]-phenol.

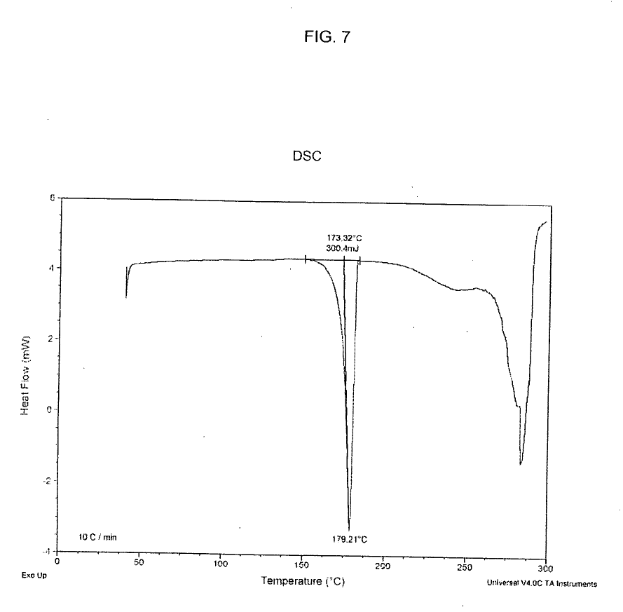

FIG. 8 provides a chart of the pH—solubility profile of 4-[2-dimethylamino-1-(cis-1-hydroxy-4-methoxy-cyclohexyl)-ethyl]-phenol.

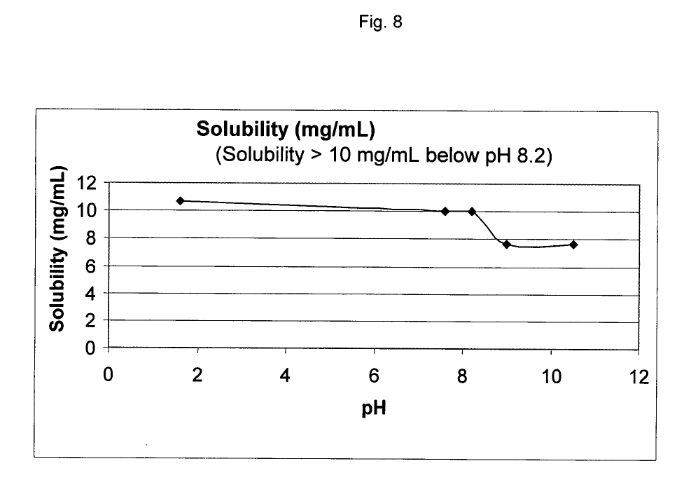

### DETAILED DESCRIPTION OF THE INVENTION

The present invention provides a new class of compounds which has the structure:

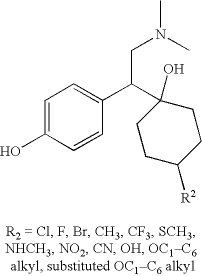

or a prodrug or a pharmaceutically acceptable salt thereof.

Advantageously, these compounds and formulations of the invention reduce the undesirable side-effects associated with many previously described SNRI's, including constipation, hypertension, and the histamine-related side-effects.

The compounds of the invention may contain one or more asymmetric carbon atoms and some of the compounds may contain one or more asymmetric (chiral) centers and may thus give rise to optical isomers and diastereomers. While shown without respect to stereochemistry in Formula (I), in one embodiment, carbon 1 is present as a chiral center. However, this molecule can exist in a form of R and S isomers as well as the racemic mixture. There are also two diastereomers. The two groups on the cyclohexane ring could be in the cis or trans configuration, but preferably in the cis configuration. For example, in one embodiment, the compound of the invention is in a configuration is greater than 50% cis diastereomer. In another embodiment, the compounds of the invention are in a configuration which is greater than 95% cis diastereomer. Thus, the invention includes such optical isomers and disastereomers; as well as the racemic and resolved, enantiomerically pure stereoisomers; as well as other mixtures of the R and S stereoisomers, and pharmaceutically acceptable salts, hydrates, and prodrugs thereof.

The term “alkyl” as a group or part of a group, e.g., alkoxy, is used herein to refer to both straight- and branched-chain saturated aliphatic hydrocarbon groups, generally of 1, 2, 3, 4, 5, 6, 7 or 8 carbon atoms in length, unless otherwise specified. The term “lower alkyl” is used to refer to alkyl chains of 1, 2, 3, or 4 carbons in length. The terms “substituted alkyl” refers to alkyl or lower alkyl as just described having from one to three substituents selected from the group including halogen, CN, OH, NO₂, amino, aryl, heterocyclic, substituted aryl, substituted heterocyclic, alkoxy, aryloxy, substituted alkyloxy, alkylcarbonyl, alkylcarboxy, alkylamino, arylthio. These substituents may be attached to any carbon of alkyl group provided that the attachment constitutes a stable chemical moiety.

The term “halogen” refers to Cl, Br, F, or I.

The term “aryl” as a group or part of a group, e.g., aryloxy, is used herein to refer to a carbocyclic aromatic system, e.g., of 6-20 carbon atoms, which may be a single ring, or multiple rings fused or linked together as such that at least one part of the fused or linked rings forms the conjugated aromatic system. The aryl groups include, but are not limited to, phenyl, naphthyl, biphenyl, anthryl, tetrahydronaphthyl, and phenanthryl.

The term “substituted aryl” refers to aryl as just defined having one, two, three or four substituents from the group including halogen, CN, OH, NO₂, amino, alkyl, cycloalkyl, alkenyl, alkynyl, alkoxy, aryloxy, substituted alkyloxy, alkylcarbonyl, alkylcarboxy, alkylamino, and arylthio.

Alkenyl and alkynyl groups may have for example 2-7 carbon atoms. Cycloalkyl groups may have 3-8 carbon atoms.

The term “heterocyclic” is used herein to describe a stable 4-, 5-, 6- or 7-membered monocyclic or a stable multicyclic heterocyclic ring which is saturated, partially unsaturated, or unsaturated, and which consists of carbon atoms and from one to four heteroatoms selected from the group including N, O, and S atoms. At least one carbon atom may be C═O. The N and S atoms may be oxidized. The heterocyclic ring also includes any multicyclic ring in which any of above defined heterocyclic rings is fused to an aryl ring. A multicyclic ring may be 2 or 3 monocyclic rings of 4- to 7-membered rings as described above. The heterocyclic ring may be attached at any heteroatom or carbon atom provided the resultant structure is chemically stable. Such heterocyclic groups include, for example, tetrahydrofuran, piperidinyl, piperazinyl, 2-oxopiperidinyl, azepinyl, pyrrolidinyl, imidazolyl, pyridyl, pyrazinyl, pyrimidinyl, pyridazinyl, oxazolyl, isoxazolyl, morpholinyl, indolyl, quinolinyl, thienyl, furyl, benzofuranyl, benzothienyl, thiamorpholinyl, thiamorpholinyl sulfoxide, and isoquinolinyl.

The term “substituted heterocyclic” is used herein to describe the heterocyclic just defined having one to four substituents selected from the group which includes halogen, CN, OH, NO₂, amino, alkyl, substituted alkyl, cycloalkyl, alkenyl, substituted alkenyl, alkynyl, alkoxy, aryloxy, substituted alkyloxy, alkylcarbonyl, alkylcarboxy, alkylamino, or arylthio.

The term “alkoxy” is used herein to refer to the OR group, where R is alkyl or substituted alkyl. The term “aryloxy” is used herein to refer to the OR group, where R is aryl or substituted aryl. The term “alkylcarbonyl” is used herein to refer to the RCO group, where R is alkyl or substituted alkyl. The term “alkylcarboxy” is used herein to refer to the COOR group, where R is alkyl or substituted alkyl. The term “aminoalkyl” refers to both secondary and tertiary amines wherein the alkyl or substituted alkyl groups, containing one to eight carbon atoms, which may be either same or different and the point of attachment is on the nitrogen atom.

The compounds of the present invention can be used in the form of salts derived from pharmaceutically or physiologically acceptable acids or bases. These salts include, but are not limited to, the following salts with organic and inorganic acids such as acetic, lactic, citric, tartaric, succinic, fumaric, maleic, malonic, mandelic, mallic, hydrochloric, hydrobromic, phosphoric, nitric, sulfuric, methanesulfonic, toluenesulfonic and similarly known acceptable acids, and mixtures thereof. Other salts include salts with alkali metals or alkaline earth metals, such as sodium (e.g., sodium hydroxide), potassium (e.g., potassium hydroxide), calcium or magnesium.

These salts, as well as other compounds of the invention may be in the form of esters, carbamates and other conventional “pro-drug” forms, which, when administered in such form, convert to the active moiety in vivo. In a currently preferred embodiment, the prodrugs are esters. See, e.g., B. Testa and J. Caldwell, “Prodrugs Revisited: The “Ad Hoc” Approach as a Complement to Ligand Design”, Medicinal Research Reviews, 16(3):233-241, ed., John Wiley & Sons (1996).

In one embodiment, the invention provides 1-[2-dimethylamino-1-(4-phenol)ethyl]-cis-1,4-cyclohexandiol, or a pharmaceutically acceptable salt, or prodrug thereof. This compound is characterized by a formula C₁₆H₂₅NO₃ and a molecular weight of about 279.38. The free base of this compound has the structure:

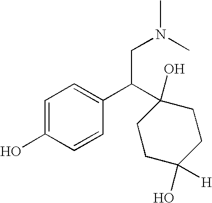

In another embodiment, the invention provides 4-[2-Dimethylamino-1-(1-hydroxy-4-propoxy-cyclohexyl)-ethyl]-phenol. This compound is characterized by a formula of C₁₇H₂₇NO₃ and a molecular weight of 293.40. The free base of this compound has the structure: 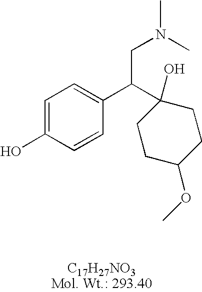
4-[2-Dimethylamino-1-(1-hydroxy-4-methoxy-cyclohexyl)-ethyl]-phenol
   Other exemplary compounds of the invention include 4-[2-Dimethylamino-1-(4-ethoxy-1-hydroxy-cyclohexyl)-ethyl]-phenol, salts and prodrugs thereof. The free base of this compound has the structure: 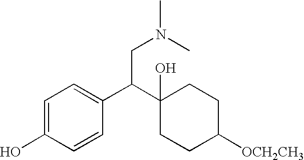
4-[2-Dimethylamino-1-(4-ethoxy-1-hydroxy-cyclohexyl)-ethyl]-phenol
   Still another exemplary compounds of the invention is 4-[2-Dimethylamino-1-(1-hydroxy-4-isopropoxy-cyclohexyl)-ethyl]-phenol, and salts and prodrugs thereof. The free base of this compound has the structure: 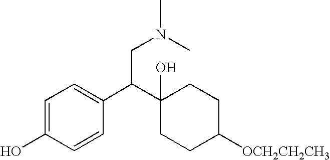
4-[2-Dimethylamino-1-(1-hydroxy-4-propoxy-cyclohexyl)-ethyl]-phenol

These and the other compounds of the invention can be prepared following the Schemes illustrated below.

Synthesis

The compounds of the present invention can be prepared using the methods described below, together with synthetic methods known in the synthetic organic arts or variations of these methods by one skilled in the art. [See, generally, Comprehensive Organic Synthesis, “Selectivity, Strategy & Efficiency in Modern Organic Chemistry”, ed., I. Fleming, Pergamon Press, New York (1991); Comprehensive Organic Chemistry, “The Synthesis and Reactions of Organic Compounds”, ed. J. F. Stoddard, Pergamon Press, New York (1979)]. Suitable methods include, but are not limited to, those outlined below.

Scheme I provides one method for the synthesis of certain compounds of the invention. A similar method can be used for synthesis of the other derivatives of the invention using different intermediates with the appropriate groups. These intermediates are commercially available.

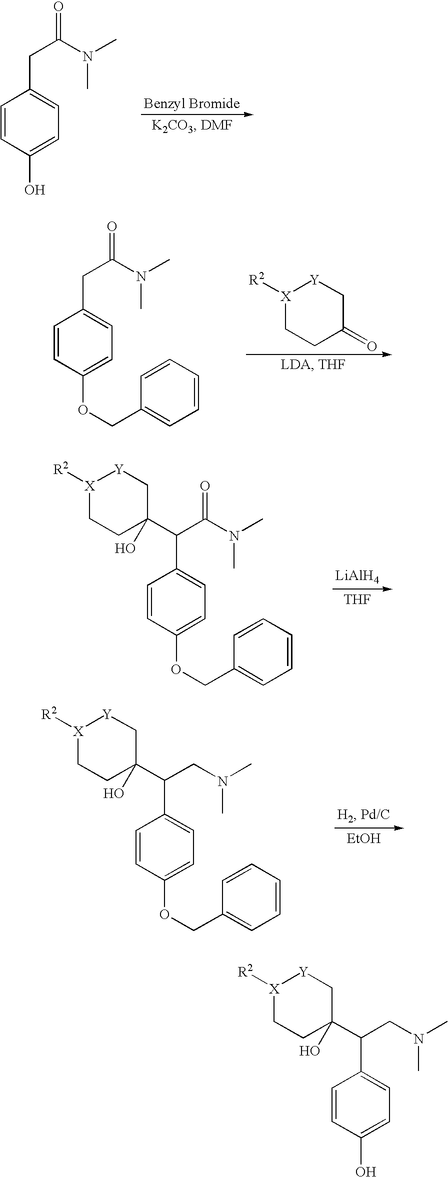

R², X and Y are as defined previously.

Alternative Synthesis

In one embodiment, the invention provides a method of preparing a compound of the structure A: 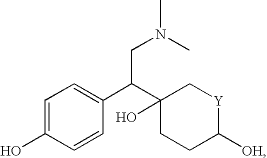
where Y is C or a bond.
or structure B: 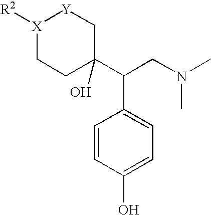
wherein X is C, N, or O; and Y is a C or absent; when X is C; R² is selected from H, halogen, CF₃, phenyl, SCH₃, NHCH₃, OC₁-C₆ alkyl, and substituted OC₁-C₆alkyl; and when X is N, R² is selected from H, phenyl or CF₃;

This method involves the step of reacting a 2-(4-hydroxy-phenol)-dimethylacetamide with a benzyl halide to afford a 2-(4-benzyloxy-phenyl)-dimethylacetamide. The 2-(4-hydroxy-phenol)-dialkylacetamide may be in a solution comprising dimethylformamide. Further, the solution can be treated with potassium carbonate prior to reaction with the benzyl halide.

To obtain the compound of structure A, the resulting 2-(4-benzyloxy-phenyl)-dimethylacetamide is subsequently reacted with a compound having the structure: 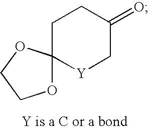
in a solution with a suitable base to afford the corresponding tertiary alcohol, ketal compound. Examples of suitable bases include, e.g., lithium diisopropylamide and isopropyl magnesium bromide. The solution (e.g., containing tetrahydrofuran (THF)) containing the ketal is reacted with an acid (e.g., aqueous HCl) and quenched to afford a ketone. The ketal hydrolysis reaction may be quenched with potassium carbonate. The resulting product is typically then extracted, concentrated, and crystallized from hot EtOAc/hexanes to afford the ketone. The ketone is reduced to selectively afford the cis diol and the amide utilizing a reducing agent selected from lithium aluminum hydride (LiAlH₄) and borane, thereby providing the corresponding dialkyl amine. In order to afford the compound of the structure A, the benzyl ether is hydrogenated to remove the benzyl group. Of course, the benzyl ether may also be removed by additional methods available to one of ordinary skill such as other reductive methods as well as acid cleavage with reagents such as HI, HBr, TMSI, etc.

To prepare the compound of structure B, the 2-(4-benzyloxy-phenyl)-dimethylacetamide is reacted with a compound having the structure: 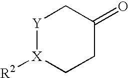
wherein X is C, N, or O; and Y is a C or absent; when X is C; R² is selected from H, halogen, CF₃, SCH₃, NHCH₃, OH, OC₁-C₆ alkyl, phenyl, and substituted OC₁—C₆alkyl; when X is N, R² is H, phenyl or CF₃; in a solution (e.g., containing THF) with a suitable base, such as described above. In one embodiment, this compound is selected from the group consisting of pyran-4-one and phenyl-piperidine-4-one. The resulting product is reduced (e.g., using LiAlH₄) to provide the corresponding dimethylamine and the benzyl ether is hydrogenated to remove the benzyl group and afford a compound of structure B.

The invention further provides useful intermediates including, e.g., a compound having the structure: 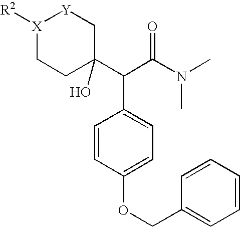
wherein R², X and Y are as defined previously; and a compound having the structure: 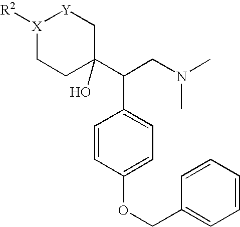
wherein R² X and Y are as defined previously, and a compound having the structure, 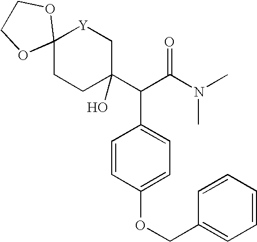
wherein Y is as defined previously, and a compound having the structure, 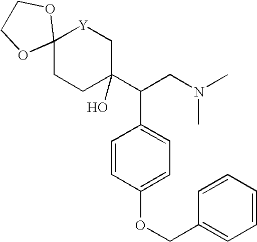
wherein Y is as defined previously, and a compound having the structure, 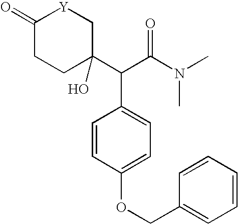
wherein Y is as defined previously, and a compound having the structure, and 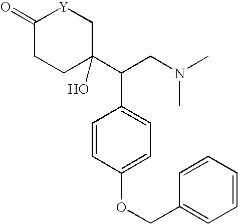
wherein Y is as defined previously.

Advantageously, it has been found that the process is highly selective for the cis-compounds, leading to a high yield and good crystallinity. Without wishing to be bound by theory, it is believed that the LAH reaction plays a significant role in this specificity.

In one embodiment, the method of synthesizing the compounds of the invention provides a compound having a configuration is greater than 50% cis diastereomer. In another embodiment, the method of synthesizing the compounds of the invention provides a compound having the configuration which is greater than 95% cis diastereomer. In another embodiment, it may be desirable to substitute sodium borohydride for the LAH.

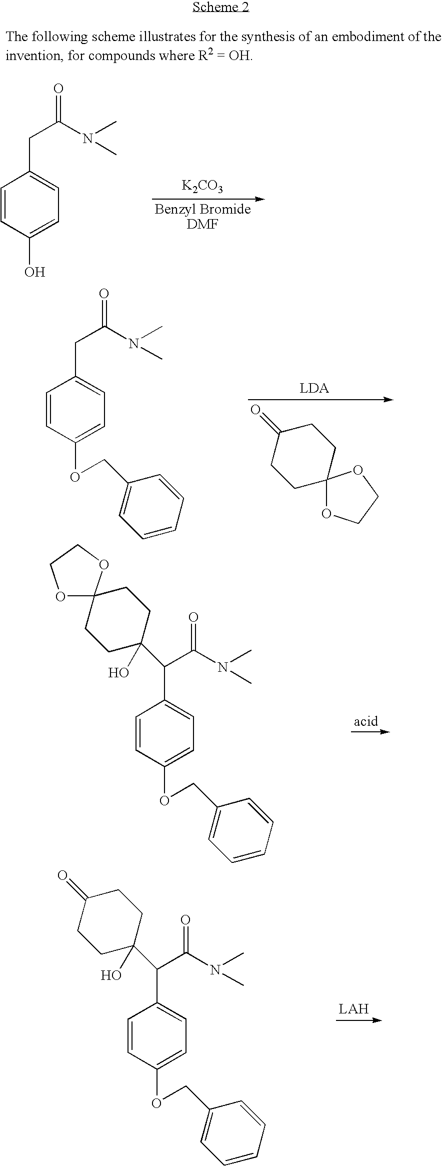

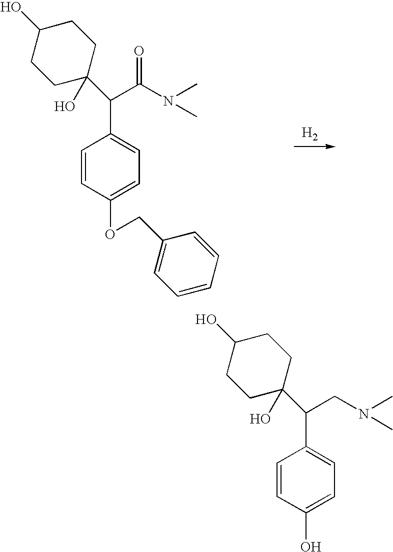

4-(Dimethylcarbamoylmethyl)phenol in dimethylformamide (DMF) is treated with K₂CO₃ followed by benzyl bromide. The benzyl bromide protecting group is particularly well suited for use in the method of synthesizing the compounds of the invention because of its ease of removal during the final step. [In an early experiment, a methyl group was used to protect the oxygen in the 4-position on the benzene ring. However, the use of L-selectride during the deprotection was difficult, leading to poor demethylation and subsequent difficulty in the LDA reaction, leading to many impurities.] However, other protecting groups may be substituted.

The mixture is stirred at room temperature followed by heating at 60° C. for 1 hour. The mixture is concentrated to remove DMF, diluted with EtOAc and washed with water. Dry MgSO₄ is added, the mixture filtered and concentrated to low volume. Hexane is added to precipitate the ketal intermediate product. Solids are collected via filtration and dryed.

A solution of the mono-ethylene ketal in 100 mL THF/50 mL MeOH is treated with acid (e.g., HCl), then stirred at room temperature. The methoxy derivative was synthesized by converting the 1,4-cyclohexanedione-mono-ethylene ketal before the LDA reaction to 4-methoxy cyclohexanone. In another embodiment, the ketal may be converted to contain the desired substituents after the LDA reaction. The ketal hydrolysis reaction is quenched with saturated K₂CO₃, extracted with EtOAc and concentrated to an oil. Product is crystallized from hot EtOAc/hexanes to provide the ketone intermediate.

A solution of the ketone in THF was added to a suspension of lithium aluminum hydride (LAH) pellets in THF at −78° C. The mixture is warmed to room temperature and stirred for at least 3 hours. The reaction is quenched with MeOH followed by 10% NaOH and stirred for at least 3 hours. The solids are removed by filtration, followed by a wash (e.g., with THF), and concentrated. The resulting solid is recrystallized from EtOAc/hexanes to provide the corresponding benzyl ether.

Advantageously, it has been found that the process is highly selective for the cis-compounds, leading to a high yield and good crystallinity. Without wishing to be bound by theory, it is believed that the LAH reaction plays a significant role in this specificity. In one embodiment, the method of synthesizing the compounds of the invention provides a compound having a configuration is greater than 50% cis diastereomer. In another embodiment, the method of synthesizing the compounds of the invention provides a compound having the configuration which is greater than 95% cis diastereomer. In another embodiment, it may be desirable to substitute sodium borohydride for the LAH.

A mixture of the benzyl ether and Pd/C in 100 mL of ethanol are hydrogenated under pressure overnight. The solid is purified by filtration followed by an ethanol wash. Solid is concentrated and crystallized from EtOAc/hexane to give the final product.

Salts may be formed by contacting stoichiometric amounts of the acid with the free base. Alternatively, the acid may be used in excess, usually no more than 1.5 equivalents. In one embodiment, the base or the acid are in solution, or both are in solution.

The crystalline salt may be prepared by directly crystallizing from a solvent. Improved yield may be obtained by evaporation of some or all of the solvent or by crystallization at elevated temperatures followed by controlled cooling, preferably in stages. Careful control of precipitation temperature and seeding may be used to improve the reproducibility of the production process and the particle size distribution and form of the product.

Use of the Compounds of Invention

The invention provides compounds with a different ratio of serotonin reuptake inhibition to norepinephrine reuptake inhibition than the currently available SNRI's. This attribute is very attractive for indications like Irritable Bowel Syndrome (IBS) where the higher NE activity of SNRI's limits the application because of constipation side effects. This lower NE activity is also attractive for patients that have cardiovascular risks related to the side effect of hypertension. It also has an application in dealing with urinary incontinence and pain.

The compositions of the present invention can be used to treat or prevent central nervous system disorders including, but not limited to, depression (including but not limited to, major depressive disorder, bipolar disorder and dysthymia), anxiety, fibromyalgia, anxiety, panic disorder, agoraphobia, post traumatic stress disorder, premenstrual dysphoric disorder (also known as premenstrual syndrome), attention deficit disorder (with and without hyperactivity), obsessive compulsive disorder (including trichotillomania), social anxiety disorder, generalized anxiety disorder, autism, schizophrenia, obesity, anorexia nervosa, bulimia nervosa, Gilles de la Tourette Syndrome, vasomotor flushing, cocaine and alcohol addiction, sexual dysfunction, (including premature ejaculation), borderline personality disorder, chronic fatigue syndrome, incontinence (including fecal incontinence, overflow incontinence, passive incontinence, reflex incontinence, stress urinary incontinence, urge incontinence, urinary exertional incontinence and urinary incontinence), pain (including but not limited to migraine, chronic back pain, phantom limb pain, central pain, neuropathic pain such as diabetic neuropathy, and postherpetic neuropathy), Shy Drager syndrome, Raynaud's syndrome, Parkinson's Disease, epilepsy, and others. Compounds and compositions of the present invention can also be used for preventing relapse or recurrence of depression; to treat cognitive impairment; for the inducement of cognitive enhancement in patient suffering from senile dementia, Alzheimer's disease, memory loss, amnesia and amnesia syndrome; and in regimens for cessation of smoking or other tobacco uses. Additionally, compounds and compositions of the present invention can be used for treating hypothalamic amenorrhea in depressed and non-depressed human females.

An effective amount of the composition of the invention is an amount sufficient to prevent, inhibit, or alleviate one or more symptoms of the aforementioned conditions. The dosage amount useful to treat, prevent, inhibit or alleviate each of the aforementioned conditions will vary with the severity of the condition to be treated and the route of administration. The dose, and dose frequency will also vary according to age, body weight, response and past medical history of the individual human patient. In generally the recommended daily dose range for the conditions described herein lie within the range of 10 mg to about 1000 mg per day, or within the range of about 15 mg to about 350 mg/day or from about 15 mg to about 140 mg/day. In other embodiments of the invention, the dosage will range from about 30 mg to about 90 mg/day. Dosage is described in terms of the free base and is adjusted accordingly for the succinate salt. In managing the patient, the therapy is generally initiated at a lower dose and increased if necessary. Dosages for non-human patients can be adjusted accordingly by one skilled in the art.

A compound of the invention may also be provided in combination with other active agents including, e.g., venlafaxine. The dosage of venlafaxine is about 75 mg to about 350 mg/day or about 75 mg to about 225 mg/day. In another embodiment, the dosage of venlafaxine is about 75 mg to about 150 mg/day. Venlafaxine or another active agent delivered in a regimen with the composition of the invention may be formulated together with the composition of the invention, or delivered separately.

Any suitable route of administration can be employed for providing the patient with an effective amount of a compound of the invention. For example, oral, mucosal (e.g., nasal, sublingual, buccal, rectal or vaginal), parental (e.g. intravenous or intramuscular), transdermal, and subcutaneous routes can be employed. Preferred routes of administration include oral, transdermal and mucosal.

A compound of the invention can be combined with a pharmaceutical carrier or excipient (e.g., pharmaceutically acceptable carriers and excipients) according to conventional pharmaceutical compounding techniques to form a pharmaceutical composition or dosage form. Suitable pharmaceutically acceptable carriers and excipients include, but are not limited to, those described in Remington's, The Science and Practice of Pharmacy, (Gennaro, A R, ed., 19th edition, 1995, Mack Pub. Co.), which is herein incorporated by reference. The phrase “pharmaceutically acceptable” refers to additives or compositions that are physiologically tolerable and do not typically produce an allergic or similar untoward reaction, such as gastric upset, dizziness and the like, when administered to an animal, such as a mammal (e.g., a human).

Compositions

In one embodiment, the composition of the invention is an immediate release formulation. In another embodiment, the composition of the invention is a sustained release formulation. Illustrative formulations are described herein. However, the invention is not so limited.

Still other suitable compositions of the invention will be readily apparent to one of skill in the art given the information provided herein. For example, in addition to providing dosing units suitable for oral administration such as tablets, capsules and caplets, the invention provides dosing units suitable for parenteral administration, transdermal or mucosal administration.

Oral solid pharmaceutical compositions may include, but are not limited to starches, sugars, microcrystalline cellulose, diluents, granulating agents, lubricants, binders and disintegrating agents. In one embodiment, the pharmaceutical composition and dosage form may also include other active components.

In one embodiment, the active component(s) are prepared in the form of a tablet or tablet-in-capsule. For example, a compound of the invention is mixed with suitable excipients to form a granulation. In one embodiment, the granulation is formed using a roller compactor. In another embodiment, the granulation is formed using a high shear granulator. However, other methods known to those of skill in the art, including, e.g., a low shear granulator, a blender, etc, can be utilized to prepare suitable granulations. The granulation is then compressed using conventional methods to form a tablet.

This tablet may be provided with additional layers, optionally, containing additional layers with active components, or other layers as may be desired for enteric coating, seal coating, separation between layers, or the like. In one embodiment, the tablet core contains one active component and a second active component is provided in a coating layer.

Optionally, a final seal coat is applied over the tablet. Suitably, this final seal coat is composed of hydroxypropylmethylcellulose (HPMC) and water, upon drying, is less than about 1 wt % of the total, coated tablet. Optionally, talc is utilized as a final step prior to filling the multi-layer tablets into a suitable packaging unit.

Alternatively or additionally, the tablet may be loaded into a capsule.

In another aspect, the invention provides a capsule containing the active component. Such capsules are produced using techniques known to those of skill in the art.

In one embodiment, the invention provides a formulation containing a core of one or more of the compounds of the invention and one or more pharmaceutically acceptable excipients, e.g., diluents, binders, fillers, glidants, anti-adherents, a pH adjuster and/or an adjuvant. The core contains about 3% w/w to about 70% w/w active compound(s). In other embodiments, the compound can range from about 5% w/w to about 60% w/w, from about 10% w/w to about 50% w/w, from about 20% w/w to about 40% w/w, or from about 25% w/w to about 35% w/w, about 30% w/w to about 45% w/w, or about 32% to about 44% w/w, based upon 100% weight of the uncoated dosage form. The core may be in a sustained release formulation or other suitable cores as are described in greater detail below may be selected. In one embodiment, a delay release coat and/or an enteric coat are provided over the core.

Suitably, the total amount of diluent, binders, fillers, glidants, anti-adherents, and adjuvants present in the core is an amount of about 30% w/w to about 97% w/w of the core, or about 25 wt % to about 80 wt % of the core. For example, when present, a binder, diluent and/or filler can each be present in an amount of about 15% w/w to about 80% w/w, or about 20% w/w to about 70% w/w, or about 25% w/w to about 45% w/w, or about 30% w/w to about 42% w/w of the uncoated dosage form. The total amount of a pH adjuster in the formulation can range from about 0.1% w/w to about 10% w/w of the core, or about 1% w/w to about 8% w/w, or about 3% w/w to about 7% w/w. However, these percentages can be adjusted as needed or desired by one of skill in the art.

The binder may be selected from among known binders, including, e.g., cellulose, and povidone, among others. In one embodiment, the binder is selected from among microcrystalline cellulose, crospovidone, and mixtures thereof.

Suitable pH adjusters include, e.g., sodium carbonate, sodium bicarbonate, potassium carbonate, lithium carbonate, among others. Still other suitable components will be readily apparent to one of skill in the art.

In one embodiment, the compound(s) of the invention is in a sustained release formulation which contains rate-controlling components. Typically, such rate controlling components are rate controlling polymers selected from among hydrophilic polymers and inert plasticized polymers. Suitable rate controlling hydrophilic polymers include, without limitation, polyvinyl alcohol (PVA), hypomellose and mixtures thereof. Examples of suitable insoluble or inert “plastic” polymers include, without limitation, one or more polymethacrylates (i.e., Eudragit® polymer). Other suitable rate-controlling polymer materials include, e.g., hydroxyalkyl celluloses, poly(ethylene) oxides, alkyl celluloses, carboxymethyl celluloses, hydrophilic cellulose derivatives, and polyethylene glycol.

In one embodiment, a formulation of the invention contains about 5% w/w to about 75% w/w microcrystalline cellulose (MCC), about 10% w/w to about 70% w/w MCC, about 20% w/w to about 60% w/w, about 25 wt % to about 30 wt %, or about 30% w/w to about 50% w/w, based on the weight of the uncoated dosage unit.

In one embodiment, the core is uncoated. These cores can be placed into a suitable capsule shell or compressed into tablets, using techniques know to those of skill in the art. Suitably, the results capsule shell or compressed tablets contain 10 mg to 400 mg of active compound.

In other embodiments, the formulation can contain one or more coatings over the core. In still other embodiments, the formulation consists of a pellet core and non-functional seal coating and a functional second coating.

In one embodiment, an initial seal coat can be applied directly to the core. Although the components of this seal coat can be modified by one of skill in the art, the seal coat may be selected from among suitable polymers such as hydroxypropyl methylcellulose (HPMC), ethylcellulose, polyvinyl alcohol, and combinations thereof, optionally containing plasticizers and other desirable components. A particularly suitable seal coat contains HPMC. For example, a suitable seal coat can be applied as a HPMC solution at a concentration of about 3% w/w to 25% w/w, and preferably 5% w/w to about 7.5% w/w. The initial seal coat can be applied on a fluid bed coater, e.g., by spraying. In one embodiment, an Aeromatic Strea™ fluid bed apparatus can be fitted with a Wurster column and bottom spray nozzle system. Approximately 200 grams of the dried pellet cores are charged into the unit. The Opadry® Clear seal coat is applied with an inlet temperature of approximately 50° C. to 60° C., a coating solution spray rate of 5 to 10 grams/minute, atomization pressure of 1 to 2 bar. Upon drying, under suitable conditions, the initial seal coat is in the range of about 1% w/w to about 3% w/w, or about 2% w/w, of the uncoated core. In another embodiment, a commercially available seal coat containing HPMC, among other inert components, is utilized. One such commercially available seal coat is Opadry® Clear (Colorcon, Inc.).

In one embodiment, the oral dosage unit contains a further release or “delay” coating layer. This release coating layer may be applied over an initial seal coat or directly over a core. In one embodiment, the release coat contains an ethylcellulose-based product and hypomellose. An example of one suitable ethylcellulose-based product is an aqueous ethylcellulose dispersion (25% solids). One such product is commercially available as Surelease® product (Colorcon, Inc.). In one embodiment, a solution of an aqueous ethylcellulose (25% solids) dispersion of about 3% w/w to about 25% w/w, and preferably about 3% to about 7%, or about 5% w/w, is applied to the core. Optionally, hypomellose, e.g., in an amount of about 5 to 15% by weight, and preferably, about 10% by weight, is mixed with the ethylcellulose dispersion, to form the coat solution. Thus, such the ethylcellulose may be about 85% to about 95%, by weight, or in embodiment, about 90% by weight, of the coat solution. Upon drying under suitable conditions, the total release coat is in the range of about 2% to about 5%, or about 3% to about 4% w/w of the uncoated or initially-coated core.

An enteric coat (rate-controlling film) may be applied to the multiparticulates and may include, but is not limited to polymethacrylates, hypomellose, and ethylcellulose, or a combination thereof. The modified release multiparticulate formulation can contain from about 3% w/w to about 70% w/w of active compound or a combination thereof, and from about 5% w/w to about 75% w/w microcrystalline cellulose, based on the weight of an uncoated dosage form.

In one embodiment, the enteric coat contains a product which is a copolymer of methacrylic acid and methacrylates, such as the commercially available Eudragit® L 30 K55 (Röhm GmbH & Co. KG). Suitably, this enteric coat is applied such that it coats the multiparticulate in an amount of about 15 to 45% w/w, or about 20% w/w to about 30% w/w, or about 25% w/w to 30% w/w of the uncoated or initially-coated multiparticulate. In one embodiment, the enteric coat is composed of a Eudragit® L30D-55 copolymer (Röhm GmbH & Co. KG), talc, triethyl citrate, and water. More particularly, the enteric coating may contain about 30% w/w of a 30 wt % dispersion of Eudragit® L 30 D55 coating; about 15% w/w talc, about 3% triethyl citrate; a pH adjuster such as sodium hydroxide and water.

In another embodiment, the enteric coat contains an ethylcellulose-based product, such as the commercially available Surelease® aqueous ethylcellulose dispersion (25% solids) product (Colorcon, Inc.). In one embodiment, a solution of Surelease® dispersion of about 3% w/w to about 25% w/w, and preferably about 3% to about 7%, or about 5% w/w, is applied to the multiparticulate. Upon drying under suitable conditions, the enteric coat is in the range of about 2% to about 5%, or about 3% to about 4% w/w of the uncoated or initially-coated core.

The enteric coat can be applied directly to the uncoated core, i.e., the uncoated core, or may be applied over an initial seal coat. The enteric coat, as described above, is typically applied on a fluid bed coater. In one embodiment, Surelease® aqueous ethylcellulose dispersion (25% solids) is applied in a similar fashion as the seal coat. After the ethylcellulose coat is applied, the core is dried for an additional 5 to 10 minutes.

In one embodiment, a final seal coat is applied over the enteric coat and, optionally, talc is utilized as a final step prior to filling the formulations into a suitable packaging unit. Suitably, this final seal coat is composed of HPMC and water, upon drying, is less than about 1 wt % of the total, coated oral dosage unit.

III. Kits

In another embodiment, the present invention provides products containing the compounds and compositions of the invention.

In one embodiment, the compositions are packaged for use by the patient or his caregiver. For example, the compositions can be packaged in a foil or other suitable package and is suitable for mixing into a food product (e.g., applesauce or the like) or into a drink for consumption by the patient.

In another embodiment, the compositions are suspended in a physiologically compatible suspending liquid. For oral liquid pharmaceutical compositions, pharmaceutical carriers and excipients can include, but are not limited to water, glycols, oils, alcohols, flavoring agents, preservatives, coloring agents, and the like.

In yet another embodiment, the compositions are filled in capsules, caplets or the like for oral delivery.

In another embodiment, the present invention provides for the use of compositions of the invention in the preparation of medicaments, including but not limited to medicaments useful in the treatment of depression, gastrointestinal side-effects of venlafaxine in a subject undergoing treatment therewith, and irritable bowel syndrome.

In another embodiment, the present invention provides for the use of multiparticulate formulations of the invention in the preparation of medicaments for delivery to a pediatric or geriatric patient.

In other embodiments, the present invention provides for the use of multiparticulate formulations of the invention in the preparation of dosing units, including but not limited to dosing units for oral, transdermal, or mucosal administration.

Also encompassed by the invention are pharmaceutical packs and kits comprising a container, such as a foil package or other suitable container, having a formulation of the invention in unit dosage form.

The following examples are illustrative of the invention.

### EXAMPLE 1

### PRODUCTION OF 1-[2-DIMETHYLAMINO-1-(4-PHENOL)ETHYL]-CIS-1,4-CYCLOHEXANDIOL

4-(Dimethylcarbamoylmethyl)phenol (35.6 g, 198.5 mmol) in dimethylformamide (DMF) (400 mL) was treated with K₂CO₃ (35.6 g, 258.0 mmol) followed by benzyl bromide (28 mL, 238 mmol). The mixture was stirred at room temperature for 4 days followed by heating at 60° C. for 1 h. The mixture was concentrated to remove DMF, diluted with EtOAc and washed with water 3×. Dry MgSO₄ was added, the mixture filtered and concentrated to low volume. Hexane was added to precipitate product. Solids were collected via filtration and dryed to give 49 g, 92% yield of a solid.

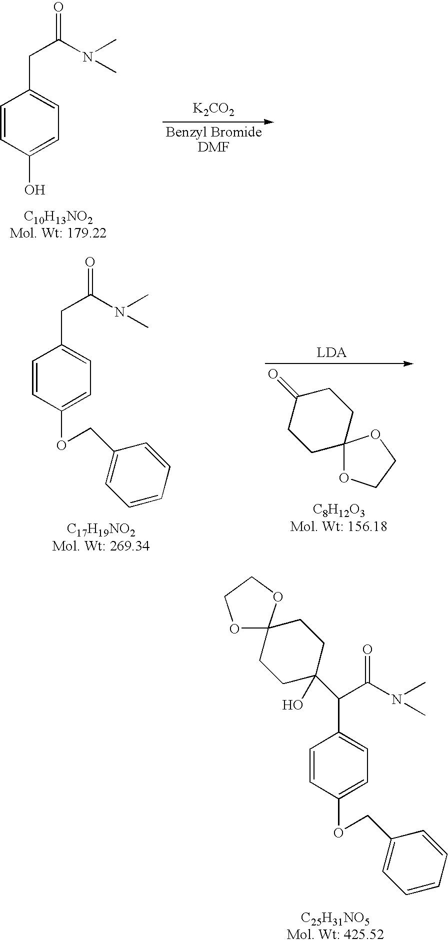

A solution of 2N lithium diisopropylamide (LDA) (48.25 mL, 96.5 mmol) was cooled to −78° C. and diluted with 25 mL of tetrahydrofuran (THF). To this was added dropwise, a solution of 2-(4-Benzyloxy-phenyl)-N,N-dimethyl-acetamide (20 g, 74.3 mmol) in 250 mL of THF. The mixture was warmed to 0° C., then cooled back to −78° C. A solution of 1,4-cyclohexanedione mono-ethylene ketal (14.1 g) in 350 mL of THF was added. The solution was allowed to warm to −20° C.

Highthroughput liquid chromatography (HPLC) assay still showed starting material. Another 1 g of ketal was added and the solution was warmed to 0° C. for 2 hour. The reaction was quenched with a mixture of 25 g NH₄Cl in 200 mL of water. EtOAc was added and the layers separated. The organic layer was dried with MgSO₄, filtered and concentrated. Column chromatography (50% EtOAc/hexanes) gave 30.3 g, 96% yield of a solid.

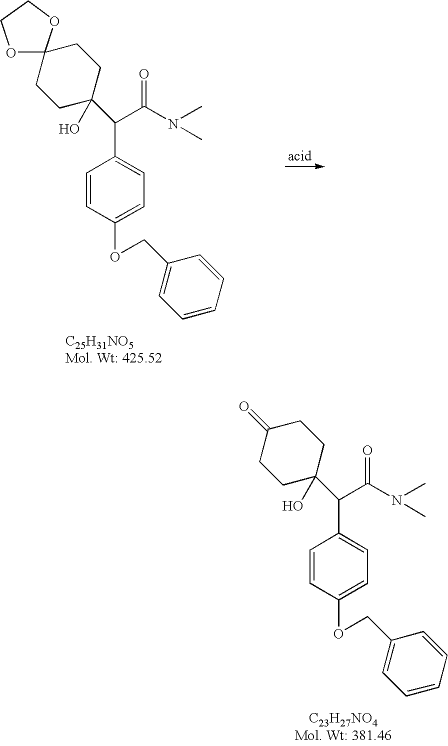

A solution of the ketal (28 g, 65.8 mmoL) in 100 mL THF/50 mL MeOH was 5 treated with 40 mL of 3N HCl, then stirred at room temperature for 3 days. The reaction was quenched with saturated K₂CO₃, extracted with EtOAc and concentrated to an oil. Product was crystallized from hot EtOAc/hexanes to provide 12.9 g, 51% yield.

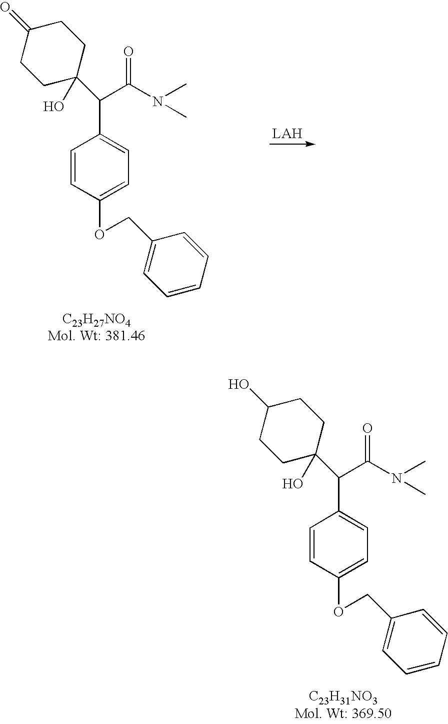

A solution of the ketone (11.8 g, 31.0 mmoL) in 100 mL of THF was added to a suspension of LAH pellets (4.7 g, 123.9 mmoL) in 100 mL of THF at −78° C. The mixture was warmed to room temperature and stirred overnight. Because starting material/intermediate was still present, another 0.75 g of lithium aluminum hydride (LAH) pellets were added and stirred for 3 h. The reaction was quenched with MeOH followed by 50 mL of 10% NaOH and stirred for 3 h. Solids were filtered off through celite, washed with THF, and concentrated. The solid was recrystallized from EtOAc/hexanes to give 8.15 g, 71% yield.

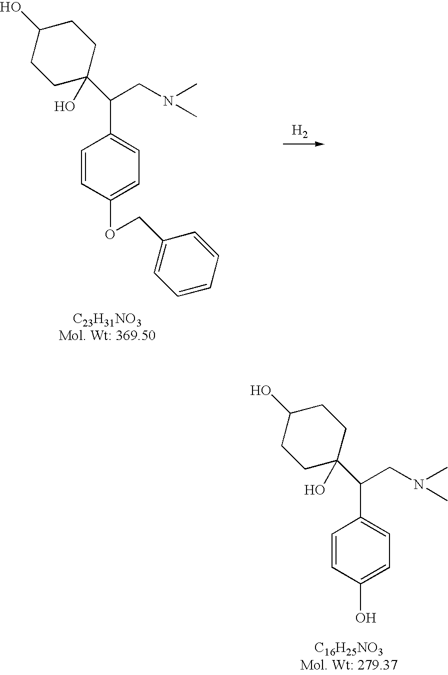

A mixture of the benzyl ether (8.1 g, 22.0 mmol) and 2.0 g of 10% Pd/C (50% wet) in 100 mL of ethanol were hydrogenated at 100 psi overnight. The mixture is filtered through celite, washed with ethanol, and concentrated. Solid product is crystallized from EtOAc/hexane to give 5.1 g, 82% yield of the title compound.

### EXAMPLE 2

### PHYSICAL-CHEMICAL PROPERTIES OF 1-[2-DIMETHYLAMINO-1-(4-PHENOL)ETHYL]-CIS-1,4-CYCLOHEXANDIOL

When prepared according to the method of Example 1, the title compound (free base) is characterized by the following:

| | |
|---|---|
| Purity | 97.51% cis-isomer, 1.91% trans-isomer, |
| | 0.22% intermediates |
| Structural Formula | 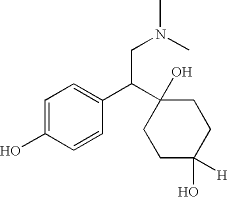 |
| Molecular Formula | C₁₆H₂₅NO₃ |
| Molecular Weight | 279.379 |
| Appearance | white to off-white crystalline powder |
| Melting point (DSC onset) | Ca. 193.3790° C. |
| X-ray (powder diff) | One polymorph |
| Hygroscopicity | Non-hygroscopic (Less than 2% weight gain |
| | at 26.30C/90% RH), weight gain is lost upon |
| | reduction in % RH) |
| Solution Stability | The compound was stable for at least 24 |
| | hours at room temperature in all of the |
| | aqueous solutions (pH 1.4-10.0). |
| pH-Solubility | Final pH 1.4 24.2 mg/ml |
| | Final pH 3.99 24.1 mg/ml |
| | Final pH 5.79 26.7 mg/ml |
| | Final pH 8.4 22.7 mg/ml |

### EXAMPLE 3

### SALT FORMS OF 1-[2-DIMETHYLAMINO-1-(4-PHENOL)ETHYL]-CIS-1,4-CYCLOHEXANDIOL

A. Succinate Salt

0.5008 g of 1-[2-dimethylamino-1-(4-phenol)ethyl]-cis-1,4-cyclohexandiol was dissolved in 3 ml of acetone. The solution was heated to 60° C. 0.206 g of succinic acid (Sigma-Aldrich), was dissolved in 7 ml of acetone with 2 drops of water and heated to 70° C. in a water bath. Succinic acid solution was added drop by drop to the 1-[2-dimethylamino-1-(4-phenol)ethyl]-cis-1,4-cyclohexandiol solution at 70° C. with mixing. Heating was continued with the addition of few drops of water to give one phase solution at 65° C. Mixing was continued at 60° C. for 10 minutes, then cooled to room temperature overnight. The precipitate that formed at the base of the flask was dissolved in ethanol and then the ethanolic solution was evaporated with a rotary evaporator under reduced pressure to give 0.4898 g of white powder.

¹H-NMR confirms the structure of the 1-[2-dimethylamino-1-(4-phenol)ethyl]-cis-1,4-cyclohexandiol succinate with a ratio of 1:1 for the 1-[2-dimethylamino-1-(4-phenol)ethyl]-cis-1,4-cyclohexandiol to the succinate.

The succinate salt has a water solubility of more than 12 mg/ml in pH's of 1.3, 4.5 and 6.5 L and is a white powder which is hygroscopic.

B. Hydrochloride Salt

0.5008 g of 1-[2-dimethylamino-1-(4-phenol)ethyl]-cis-1,4-cyclohexandiol was dissolved in 10 mL of acetone with 10 drops of water and heated in a water bath to 70° C. to give a clear solution. 2 g of 1N hydrochloric acid was heated to 70° C. and added drop by drop to the 1-[2-dimethylamino-1-(4-phenol)ethyl]-cis-1,4-cyclohexandiol solution. The resulted solution was kept mix at 70° C. for 30 minutes. All solvents were evaporated with rotary evaporator under reduced pressure to give a yellow-pink solid. The later was dissolved in ethanol and the ethanolic solution was evaporated with rotary evaporator under reduced pressure to give 0.4894 g of off-white solid. H-NMR confirms the structure of the 1-[2-dimethylamino-1-(4-phenol)ethyl]-cis-1,4-cyclohexandiol hydrochloride with a ratio of 1:1 for the 1-[2-dimethylamino-1-(4-phenol)ethyl]-cis-1,4-cyclohexandiol to the hydrochloride.

### EXAMPLE 4

### PRODUCTION OF 4-[2-DIMETHYLAMINO-1-(CIS-1-HYDROXY-4-METHOXY-CYCLOHEXYL)-ETHYL]-PHENOL

The compound was prepared in a similar pathway as that of Example 1 with some minor changes including the use of 4-methoxy-cyclohexanone to obtain a 4-methoxy on the cyclohexanol. The synthesis summary for 4-methoxy-cyclohexanone is described below. The remaining synthesis steps were completed as described in Example 1.

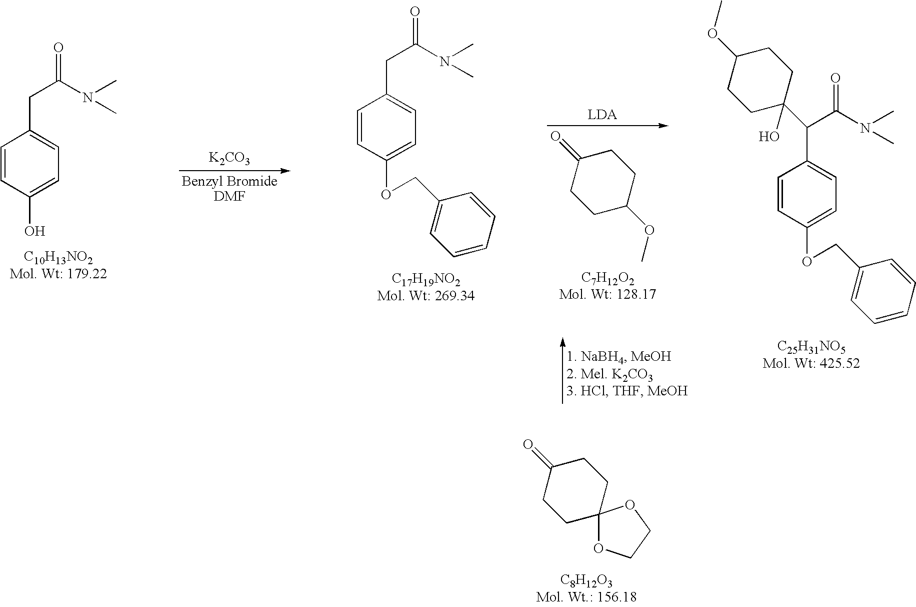

### EXAMPLE 5

### PHYSICAL-CHEMICAL PROPERTIES OF 4-[2-DIMETHYLAMINO-1-(CIS-1-HYDROXY-4-M ETHOXY-CYCLOHEXYL)-ETHYL]-PHENOL

When prepared according to the method of Example 4, the title compound (free base) is characterized by the following:

| | |
|---|---|
| Purity | 99% |
| Structural Formula | 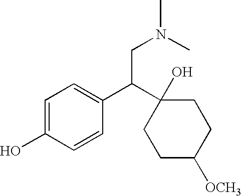 |
| Molecular Formula | C₁₇H₂₇NO₃ |
| Molecular Weight | 293.40 |
| Appearance | white crystalline powder |
| Melting point (DSC onset) | 179.21° C. |
| X-ray (powder diff) | Crystalline-one-polymorph |
| Hygroscopicity | Non-hygroscopic (0.44% weight gain @ |
| | 60% RH, 1.2% weight gain @ 90% RH, |
| | weight gain was lost when returned to |
| | 10% RH or 0% RH.) |
| Solution Stability | The compound was stable for at least 72 |
| | hours at room temperature in all of the aque- |
| | ous solutions (pH 1.6-10.5) |
| pH-Solubility | Final pH 1.60 >10.71 mg/ml |
| | Final pH 7.62 >10.00 mg/ml |
| | Final pH 8.21 >10.00 mg/ml |
| | Final pH 9.00 7.65 mg/ml |
| | Final pH 10.5 7.65 mg/ml |
| Octanol/Water | C_{oct}/C_{aq} @ pH 6 = 6.857 |
| Partitioning Coefficient | |

### EXAMPLE 6

### PERMEABILITY ASSESSMENT OF FREE BASE AND SALT FORMS OF 1-[2-DIMETHYLAMINO-1-(4-PHENOL)ETHYL]-CIS-1,4-CYCLOHEXANDIOL-HTS-24 CACO-2 MODEL

The rate of drug transport through the CACO-2 cells was determined as the Apparent Permeability Coefficient according to the following formula:

Papp=AQ X Rv cm.s- AT 60.A.Co

ΔQ=Change in quantity

ΔT=Change in time (minutes)

Cₒ=Initial concₙ in the donor chamber (mM.cm⁻³)

A=Surface area of membrane (cm²)

60=Conversion factor to give cm.s⁻¹

Rv=volume of receiver compartment.

Transepithelial electrical resistance (TER) was calculated from resistance measurements according to the following formula: TER=(R[cells+filter+medium])−(R[filter+medium])×cell area.

Apparent permeability rates were interpreted as follows. Apparent permeability values which are equal to or greater than those observed for metoprolol or propranolol during the same assay run are considered to give a predicted fraction absorbed estimate of ≧90% (high permeability classification). Apparent permeability values less than metoprolol or propranolol are considered to be ≦90% fa (moderate permeability classification). Apparent permeability values of <10 nms⁻¹ are considered to be ≦50% fa (low permeability classification). TER values of <120 ohms cm² indicate low monolayer integrity over the assay period.

A compound/metoprolol or propranolol ratio of ≧1 indicates a high permeability compound. A compound/metoprolol or propranolol ratio of <1 indicates a moderate to low permeability compound.

Compound/Propranolol ratio:
Free Base=2.9
HCl salt=2.9
Succinate salt=2.9

Thus, the free base and both tested salts of this compound are highly permeable.

Permeability Conclusion:

There was no difference in permeability between the base and salt forms (HCl and succinate) for 1-[2-dimethylamino-1-(4-phenol)ethyl]-cis-1,4-cyclohexandiol under these caco-2 assay test conditions. Predicted permeability in the GI tract was higher than that observed for the propranolol reference compound, indicating 1-[2-dimethylamino-1-(4-phenol)ethyl]-cis-1,4-cyclohexandiol is predicted as a high permeability classified compound. In each case B to A compound directional transport was lower and the B:A ratio was calculated at 0.4:1 indicating no efflux activity. Percent compound recovery was good to being slightly high in either flux direction assays. Filter control compound flux was high, with good recovery of compound. This suggested there was no evidence of degradation or metabolism in this assay system.

### EXAMPLE 7

### PHARMACOLOGY FOR 1-[2-DIMETHYLAMINO-1-(4-PHENOL)ETHYL]-CIS-1,4-CYCLOHEXANDIOL

The following table is a summary of receptor assay binding studies conducted for 1-[2-dimethylamino-1-(4-phenol)ethyl]-cis-1,4-cyclohexandiol (Test Compound). These assays were prepared as described in the following publications, as modified by Novascreen. The receptor binding assays were Adrenergic α-2A (human) binding assay [D. B. Bylund et al, J Pharmacol & Exp Ther, 245(2):600-607 (1988), with modifications; J A Totaro et al, Life Sciences, 44:459-467 (1989)]; dopamine transporter binding assay [Madras et al, Mol. Pharmacol., 36:518-524, with modifications, J J Javitch et al, Mol Pharmacol, 26:35-44 (1984)]; histamine H1 binding assay [Chang, et al., J Neurochem, 32:1658-1663 (1979), with modifications, J I Martinez-Mir, et al., Brain Res, 526:322-327 (1990); E E J Haaksma, et al, Pharmacol Ther, 47:73-104 (1990)]; imidazoline binding assay [C M Brown et al, Brit. J Pharmacol, 99(4):803-809 (1990), with modifications], muscarinic M5 (human recombinant) binding assay [N J Buckley et al, Mol Pharmacol, 35:469-476 (1989), with modifications]; norepinephrine transporter (human recombinant) binding assay [R. Raisman, et al., Eur J Pharmacol, 78:345-351 (1982), with modification, S. Z. Raisman, et al, Eur J Pharmacol, 72:423 (1981)]; serotonin transporter (human) binding assay [R J D'Amato, et al, J Pharmacol & Exp Ther, 242:364-371 (1987), with modifications; N L Brown et al, Eur J Pharmacol, 123:161-165 (1986)]. The cellular/functional assays were the norepinephrine transport (NET-T) human [A. Galli, et al, J Exp Biol, 198:2197-2212 (1995); and the serotonin transport (Human) assay [D'Amato et al, cited above and NL Brown et al, Eur J Pharmacol, 123:161-165 (1986)]. The results are shown in % Inhibition of the receptor.

| | Receptor Binding |
|---|---|
| | % Inhibition |
| Receptor Binding Assay | Test Compound @ 10 1 M |
| Adenosine A1 | −8.51 |
| Adenosine A2A (hr) | 7.68 |
| Adrenergic, Alpha 1A | −1.96 |
| Adrenergic, Alpha 1B | 11.27 |
| Adrenergic, Alpha 2A (h) | 10.45 |
| Adrenergic, Alpha 2B | 8.60 |
| Adrenergic, Alpha 2C (hr) | 3.06 |
| Adrenergic, Beta 1 (hr) | 7.65 |
| Adrenergic, Beta 2 (hr) | −2.46 |
| Benzodiazepine, peripheral (h) | 1.65 |
| Cannabinoid, CB1 (hr) | 8.11 |
| Cannabinoid, CB2 (hr) | 15.66 |
| Dopamine Transporter | 8.49 |
| Dopamine, D1 (hr) | −5.73 |
| Dopamine, D2s (hr) | 6.17 |
| Dopamine, D3 (hr) | 3.36 |
| Dopamine, D4.4 (hr) | 3.39 |
| GABA A, Agonist site | −16.06 |
| GABA A, Benzo, central | 0.20 |
| Glutamate, AMPA Site | 2.50 |
| Glutamate, Kainate Site | −0.51 |
| Glutamate, NMDA agonist Site | −2.99 |
| Glutamate, NMDA Glycine | 2.24 |
| Glycine, Strychnine-sensitive | −2.38 |
| Histamine, H1 | −7.84 |
| Histamine, H3 | 14.00 |
| Imidazoline, I1 | 17.43 |
| Imidazoline, I2 central | 3.24 |
| Melatonin | 11.20 |
| Muscarinic, M1 (hr) | −5.96 |
| Muscarinic, M2 (hr) | 11.98 |
| Muscarinic, M3 (hr) | 0.11 |
| Muscarinic, M4 (hr) | −3.52 |
| Muscarinic, MS (hr) | 5.40 |
| Nicotinic (a-bungarotoxin insens) | −1.07 |
| Norepinephrine transporter | 49.72 |
| Opiate, Delta 2 (hr) | 12.23 |
| Opiate, Kappa (hr) | −4.17 |
| Opiate, Mu (hr) | −0.35 |
| Oxidase, MAO-A, Central | −0.11 |
| Oxidase, MAO-A, Central | 2.43 |
| Serotonin Transporter | 94.45 |
| Serotonin, 5HT1, non-selective | 5.09 |
| Serotonin, 5HT1A (hr) | 1.80 |
| Serotonin, 5HT1D (h) | 7.26 |
| Serotonin, 5HT2A (h) | 3.70 |
| Serotonin, 5HT2C | 10.05 |
| Serotonin, 5HT3 (hr) | 9.72 |
| Serotonin, 5HT4 | 3.98 |
| Serotonin, 5HT5A (hr) | 19.95 |
| Serotonin, 5HT6 (hr) | 17.05 |
| Sigma 1 | 2.96 |
| Sigma 2 | 1.00 |

−20% to 20% Baseline - no activity at the receptor
21% to 49% - Marginal activity at the receptor
>50% - Compound is active at the receptor

From this data it is evident that 1-[2-dimethylamino-1-(4-phenol)ethyl]-cis-1,4-cyclohexandiol has very good serotonin reuptake inhibition activity and acceptable norepinephrine reuptake inhibition activity. It is also evident that 1-[2-dimethylamino-1-(4-phenol)ethyl]-cis-1,4-cyclohexandiol is highly selective in that it has no significant binding to other receptors that are usually associated with specific side effects, such as dry mouth and drowsiness (muscarinic/cholinergic), sedation or appetite-stimulation (Histamine H1) and cardiovascular effects (alpha-adrenergic).

These conclusions are based upon Novascreen's interpretation summarized above.

### EXAMPLE 8

### PHARMACOKINETICS AND METABOLISM FOR 1-[2-DIMETHYLAMINO-1-(4-PHENOL)ETHYL]-CIS-1,4-CYCLOHEXANDIOL

These studies were conducted to determine the potential metabolism of this compound in humans. These results indicate that the metabolism in humans will not be very significant. This is an advantage for this compound, because it has good systemic exposure, so almost all the drug that remains in the body is the actual compound and not a metabolite.

A. In-Vitro Metabolism

In vitro metabolism of 1-[2-dimethylamino-1-(4-phenol)ethyl]-cis-1,4-cyclohexandiol was conducted in the hepatic microsomes of Sprague-Dawley rats, male dogs, male monkeys and mixed male and female humans to characterize metabolic stability and identification of metabolites. 1-[2-dimethylamino-1-(4-phenol)ethyl]-cis-1,4-cyclohexandiol was stable (t_{1/2}>60 minutes) in hepatic microsomes indicating Phase I and II metabolism was minimal in all species.

Based on LC/MS analysis, 1-[2-dimethylamino-1-(4-phenol)ethyl]-cis-1,4-cyclohexandiol appeared to be very stable under experimental condition as only one minor metabolite, N-demethylation, was detected in all species. The proposed in vitro metabolite pathway of 1-[2-dimethylamino-1-(4-phenol)ethyl]-cis-1,4-cyclohexandiol is shown below.

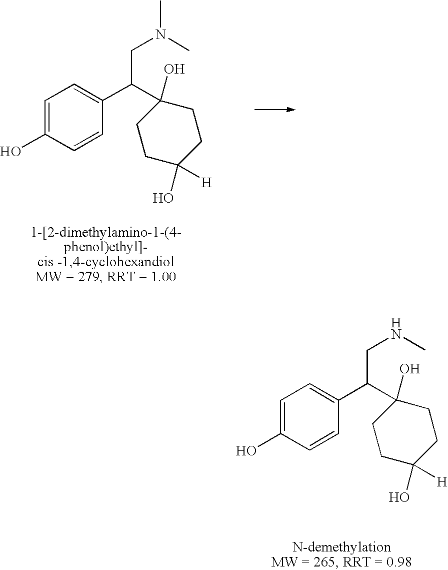

B. Pharmacokinetics in Dog

Preclinical pharmacokinetics of 1-[2-dimethylamino-1-(4-phenol)ethyl]-cis-1,4-cyclohexandiol was determined after a single 2.5 mg/kg IV bolus and 7.5 mg/kg oral dose in male dogs. After an IV dose (Water for injection at 0.1 mL/kg) in male dogs, plasma clearance was low (˜7 mL/min/kg compared with a hepatic blood flow of ˜38 mL/min/kg) and was consistent with in vitro hepatic intrinsic clearance. The apparent volume of distribution (V_{SS}) was moderate (1.9 L/kg) and the apparent terminal half-life (t_{1/2}) was long (5.6 hour). After a single oral dose of 7.5 mg/kg (Water at 1 mL/kg), the terminal oral half-life was of long duration (6 hr), however it was similar to the IV elimination half-life suggesting that the rate controlling step after oral administration was the elimination of the drug. The oral bioavailability was high (60%).

### EXAMPLE 9

### IN-VIVO EFFICACY OF 1-[2-DIMETHYLAMINO-1-(4-PHENOL)ETHYL]-CIS-1,4-CYCLOHEXANDIOL IN MICRODIALYSIS MODEL

1-[2-dimethylamino-1-(4-phenol)ethyl]-cis-1,4-cyclohexandiol was evaluated in a microdialysis study conducted in male Sprague-Dawley rats [M T Taber et al, Differential effects of coadministration of fluoxetine and WAY-100635 on serotonergic neurotransmission in vivo: sensitivity to sequence of injections, Synapse, 2000 October; 38(1):17-26.] This technique can capture the neurochemical effects of compounds in the brains of freely-moving rodents. The effects of 1-[2-dimethylamino-1-(4-phenol)ethyl]-cis-1,4-cyclohexandiol were studied in the rat dorsal lateral frontal cortex, a brain region thought to be involved in etiology and/or treatment of depression. To see whether any effects on serotonin could be observed, the compound (30 mg/kg, sc) was tested in combination with the selective 5-HT1A antagonist, N-[2-[4-(2-methoxyphenyl)-1-piperazinyl] ethyl]-N-(2-pyridinyl)cyclohexanecarboxamide (WAY-100635). The rationale for doing this is to block the somatodendritic 5-HT1A autoreceptors regulating 5-HT release. This eliminates the need to perform a chronic (14 day) neurochemical study with the compound alone to desensitize the 5-HT1A receptors. The conditions of the study are listed below:

Animal: Male Sprague-Dawley rats (280-350 g)

Brain Region: Dorsal Lateral (DL) Frontal Cortex (A/P+3.2 mm, M/L±3.5 mm, D/V −1.5 mm)

Administration:

24 hr post-operative recovery
3 hr equilibration after probe insertion
1 hr 40 min baseline
5-HT_{1A} antagonist N-[2-[4-(2-methoxyphenyl)-1-piperazinyl]ethyl]-N-(2-pyridinyl)cyclohexanecarboxamide [WAY-100635] (0.3 mg/kg, s.c.) given 20 min before 1-[2-dimethylamino-1-(4-phenol)ethyl]-cis-1,4-cyclohexandiol (30 mg/kg, po)
   Sample Collection: Samples collected for 3 hr 2 min post-injections
   Analysis: 5-HT levels quantified by HPLC-ECD

Robust elevations in cortical 5-HT were observed when 1-[2-dimethylamino-1-(4-phenol)ethyl]-cis-1,4-cyclohexandiol was combined with a 5-HT1A antagonist. These in vivo neurochemical effects are similar to observed effects when combining other SNRIs and SSRIs like venlafaxine and fluoxetine with 5-HT1A antagonism. These in-vivo results corroborate the in-vitro pharmacological profile for this compound.

### EXAMPLE 10

### PRE-CLINICAL EFFICACY OF 1-[2-DIMETHYLAMINO-1-(4-PHENOL)ETHYL]-CIS-1,4-CYCLOHEXANDIOL IN ANIMAL MODELS FOR PAIN

Current SNRIs have been shown to have some effects for various pain indications. 1-[2-dimethylamino-1-(4-phenol)ethyl]-cis-1,4-cyclohexandiol was evaluated in two in-vivo animal models for pain, including a Visceral Pain model and a Neuropathic Pain model. The compound was found to cause a statistically significant reversal of visceral pain in the mouse PPQ induced writhing model at the highest dose tested (100 mg/kg) and a statistically significant reversal of mechanical hyperalgesia in the spinal nerve ligation model of neuropathic pain in the rate (MED, 10 mg/kg). 1-[2-dimethylamino-1-(4-phenol)ethyl]-cis-1,4-cyclohexandiol was not found to affect acute pain in either the rat hot plate or tail flick assays (up to 30 mg/kg po) and was not found to reverse tactile allodynia in the spinal nerve ligation model of neuropathic pain in the rat (up to 100 mg/kg) in at the doses tested.

A. Compound Administration:

1-[2-dimethylamino-1-(4-phenol)ethyl]-cis-1,4-cyclohexandiol was dissolved in sterile saline and administered orally (p.o.) at dosages of 10 mg/kg, 30 mg/kg and 100 mg/kg. Gabapentin was purchased from Toronto Research Chemicals (Ontario, Canada) and suspended in 2% Tween 80 in 0.5% methylcellulose and administered intraperitoneally (i.p.).

B. Subjects:

For the visceral pain study male CD-1 mice (20-25 g, Charles River; Kingston/Stoneridge, NY) were housed in groups of 5/cage on bedding and for the neuropathic study male Sprague-Dawley rats (125-150 g, Harlan; Indianapolis, Ind.) were housed 3/cage on bedding. For all studies animals were maintained in climate-controlled rooms on a 12-hour light/dark cycle (lights on at 0630) with food and water available ad libitum.

C. Visceral Pain Model: Assessment of PPQ-Induced Constrictions (Writhing):

The ability of 1-[2-dimethylamino-1-(4-phenol)ethyl]-cis-1,4-cyclohexandiol to attenuate acute visceral (abdominal) pain was assessed following an i.p. injection of 2 mg/kg PPQ (dissolved in 4% ethanol in distilled water, Sigma-Aldrich, St. Louis, Mo.) [Siegmund, E., et al., Proceedings of the Society for Experimental Biology and Medicine., 95 (1957) 279-731]. The compound was pretreated 60 minutes (n=7-10/group) prior to PPQ administration. During testing, following PPQ administration, mice were individually placed in a Plexiglas cage and the total number of abdominal constrictions was recorded for one-minute periods, starting at 5 and 10 minutes after PPQ injection.

Statistical significance was determined using a one-way ANOVA using a customized SAS-excel application (SAS Institute, Cary, N.C.). Significant main effects were analyzed further by subsequent least significant difference analysis. The criterion for significant differences was p<0.05 compared to vehicle-treated mice.

A positive result (reduction in writhing) was found for 1-[2-dimethylamino-1-(4-phenol)ethyl]-cis-1,4-cyclohexandiol at 10 and 30 mg/kg, was not statistically significant. A significant reduction in writhing was obtained at 100 mg/kg dosage.

D. Neuropathic Model:

1. L5 Spinal Nerve Ligation (SNL):

Surgical procedures were performed under 4% isoflurane/O₂ anesthesia, delivered via nose cone and maintained at 2.5% for the duration of the surgery. After induction of anesthesia, the incision site was shaved and prepared in a sterile manner. Spinal nerve ligation (SNL) surgery was performed as previously described [Kim, S. H. and Chung, J. M., An experimental model for peripheral neuropathy produced by segmental spinal nerve ligation in the rat, Pain, 50 (1992) 355-63] with the exception that nerve injury was produced by tight ligation of the left L5 spinal nerve. Briefly, a midline incision was made and the L5 transverse process was removed and the nerve was tightly ligated with 6-0 silk suture material and the wound was closed in layers with 4-0 vicryl suture and the skin closed with wound clips.

For the neuropathic model statistical significance was determined using a repeated measure ANOVA using a customized SAS-excel application (SAS Institute, Cary, N.C.). Significant main effects were analyzed further by subsequent least significant difference analysis. The criterion for significant differences was p<0.05 compared to vehicle-treated rats. A positive trend was observed for 1-[2-dimethylamino-1-(4-phenol)ethyl]-cis-1,4-cyclohexandiol at 10 and 30 mg/kg versus the SNL/vehicle.

The positive results for 1-[2-dimethylamino-1-(4-phenol)ethyl]-cis-1,4-cyclohexandiol in pre-clinical spinal nerve ligation models for neuropathic pain indicate the potential for this compound as a therapy for pain indications including but not limited to visceral and neuropathic pain.

2. Behavioral Testing:

Assessment of mechanical hyperalgesia thresholds were measured as the hind paw withdrawal threshold to a noxious mechanical stimulus and was determined using the paw pressure technique [Randall, L. O. and Selitto, J. J., A method for measurement of analgesic activity on inflamed tissue, Arch. Int. Pharmacodyn. 3 (1957) 409-419]. The analgesymeter (7200, Ugo Basile, Italy) employs a rounded probe applied to the dorsum of the hind paw, cutoff was set at 250 g and the endpoint was taken as paw withdrawal. Thresholds were evaluated prior to surgery and reassessed three weeks after SNL surgery. On test day, rats were administered vehicle or test compound mechanical thresholds assessed 1, 3, 5 and 24 hr after administration (n=10/group).

The present invention is not to be limited in scope by the specific embodiments described herein. Various modifications to these embodiments will be obvious to one of skill in the art from the description. Such modifications fall within the scope of the appended claims.

Patents, patent applications, publications, procedures and the like are cited throughout the application. These documents are incorporated by reference herein.

1. A compound of the structure: 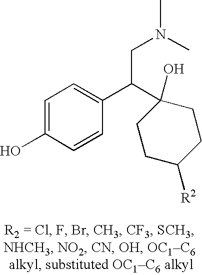
or a prodrug or a pharmaceutically acceptable salt thereof.

2. The compound according to claim 1, wherein R² is OH, or a prodrug or pharmaceutically acceptable salt thereof.

3. The compound according to claim 1, wherein R² is O-methyl.

4. The compound according to claim 1, wherein R² is O-ethyl.

5. The compound according to claim 1, wherein R² is O-propyl or isopropyl.

6. The compound according to claim 1, wherein the prodrug is an ester, ether, or carbamate of said compound.

7. The compound according to claim 1, wherein the pharmaceutically acceptable salt is selected from a hydrochloride, succinate or formate salt.

8. The compound according to claim 1 comprising greater than 50% cis diastereomer

9. The compound according to claim 1 comprising greater than 95% cis diastereomer.

10. A pharmaceutical composition comprising a compound according to claim 1 or a prodrug or a pharmaceutically acceptable salt thereof and a pharmaceutically acceptable carrier.

11. The pharmaceutical composition according to claim 10, wherein said compound is selected from the group consisting of
1-[2-Dimethylamino-1-(4-phenol)ethyl]-cis-1,4-cyclohexandiol;
4-[2-dimethylamino-1-(cis-1-hydroxy-4-methoxy-cyclohexyl)-ethyl]-phenol;
4-[2-Dimethylamino-1-(4-ethoxy-1-hydroxy-cyclohexyl)-ethyl]-phenol; and
4-[2-Dimethylamino-1-(1-hydroxy-4-propoxy-cyclohexyl)-ethyl]-phenol; and
4-[2-Dimethylamino-1-(1-hydroxy-4-isopropoxy-cyclohexyl)-ethyl]-phenol,
or a prodrug or a pharmaceutically acceptable salt thereof.

12. The pharmaceutical composition according to claim 10, comprising an oral dosage unit.

13. The pharmaceutical composition according to claim 12, wherein said oral dosage unit is a capsule or tablet.

14. The pharmaceutical composition according to claim 10, comprising an immediate release formulation.

15. The pharmaceutical composition according to claim 10, comprising a sustained release formulation.

16. A method of treating irritable bowel syndrome comprising administering a compound according to claim 1 or a prodrug or a pharmaceutically acceptable salt thereof, to a subject in need thereof.

17. A method of treating pain or pain syndromes comprising administering a compound according to claim 1 to a subject in need thereof.

18. The method according to claim 17, wherein the pain is selected from visceral and neuropathic pain or pain syndromes.

19. A method of treating urinary incontinence comprising administering a compound according to claim 1, or a prodrug or a pharmaceutically acceptable salt thereof, to a subject in need thereof.

20. A method of treating depression, fibromyalgia, anxiety, panic disorder, agoraphobia, post traumatic stress disorder, premenstrual dysphoric disorder, attention deficit disorder, obsessive compulsive disorder, social anxiety disorder, generalized anxiety disorder, autism, schizophrenia, obesity, anorexia nervosa, bulimia nervosa, Gilles de la Tourette Syndrome, vasomotor flushing, cocaine and alcohol addiction, sexual dysfunction, borderline personality disorder, fibromyalgia syndrome, diabetic neuropathic pain, chronic fatigue syndrome, Shy Drager syndrome, Raynaud's syndrome, Parkinson's Disease, and epilepsy, said method comprising administering a therapeutically effective amount of a compound according to claim 1, or a prodrug or a pharmaceutically acceptable salt thereof.

21. The method of claim 20, wherein the depression is major depressive disorder (MDD).

22. The method of claim 20, wherein the sexual dysfunction is premature ejaculation.

23. The method according to claims 16, wherein the compound is formulated for once a day dosing.

24. A method of preparing a compound of the structure (A): 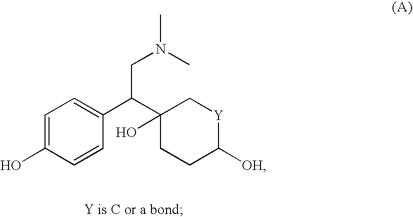
said method comprising the steps of:
(a) reacting a 2-(4-hydroxyphenol)-dimethylacetamide with a benzyl halide to afford a 2-(4-benzyloxy-phenyl)-dimethylacetamide;
(b) reacting the 2-(4-benzyloxyphenyl)-dimethylacetamide with a compound having the structure: 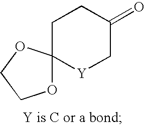
in a solution with a suitable base to afford the corresponding ketal compound;
(c) reacting the solution containing the ketal with an acid to afford a ketone;
(d) selectively reducing the ketone to afford the cis diol and the amide utilizing a reducing agent selected from lithium aluminum hydride and borane, thereby providing the corresponding dimethyl amine; and
(e) hydrogenating the benzyl ether to remove the benzyl group and afford the compound of structure (A).

25. The method according to claim 24(c), wherein the acid is aqueous HCl.

26. The method according to claim 24(c), wherein the reaction is quenched with potassium carbonate, extracted, concentrated, and crystallized from hot EtOAc/hexanes to afford the ketone.

27. A method of preparing a compound of the structure (B): 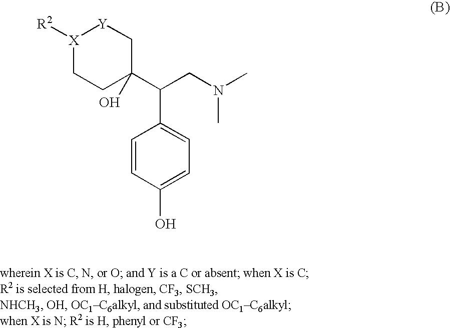
(B)
said method comprising the steps of:
(a) reacting a 2-(4-hydroxyphenol)-dimethylacetamide with a benzyl halide to afford a 2-(4-benzyloxyphenyl)-dimethylacetamide;
(b) reacting the 2-(4-benzyloxyphenyl)-dimethylacetamide with a compound having the structure: 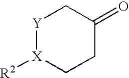
wherein X is C, N, or O; and Y is a C or absent; when X is C; R² is selected from H, halogen, CF₃, SCH₃, NHCH₃, OH, OC₁-C₆ alkyl, phenyl, and substituted OC₁-C₆alkyl; when X is N, R² is H, phenyl or CF₃;
in a solution with a suitable base;
(c) reducing the product of (b) to provide the corresponding dimethylamine;
(d) hydrogenating the benzyl ether to remove the benzyl group and afford the compound of structure (B).

28. The method according to claim 27(b), wherein the compound having the structure 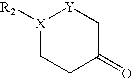
is selected from the group consisting of pyran-4-one and phenyl-piperidine-4-one.

29. The method according to claim 27, wherein the 2-(4-hydroxy-phenol-dialkylacetamide in step (a) is in a solution comprising dimethylformamide.

30. The method according to claim 29, wherein the solution is treated with potassium carbonate prior to reaction with the benzyl halide.

31. The method according to claim 27, wherein the compound in step (b) is in a solution comprising tetrahydrofuran.

32. The method according to claim 27, wherein the reduction is performed utilizing lithium aluminum hydride.

33. The method according to any one of claim 27, wherein the base is selected from the group consisting of lithium diisopropylamide and isopropyl magnesium bromide.

34. A compound having the structure: 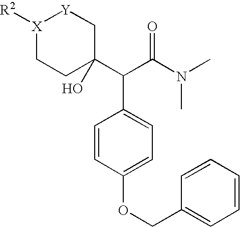
wherein X is C, N, or O; and Y is a C or absent; when X is C; R² is selected from H, halogen, CF₃, SCH₃, NHCH₃, OH, OC₁-C₆ alkyl, phenyl, and substituted OC₁-C₆ alkyl; when X is N; and R₂ is H, phenyl or CF₃.

35. A compound having the structure: 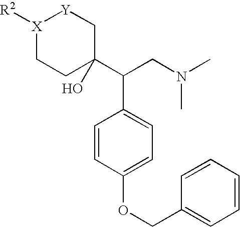
wherein X is C, N, or O; and Y is a C or absent; when X is C; R² is selected from H, halogen, CF₃, SCH₃, NHCH₃, OH, OC₁-C₆ alkyl, phenyl, and substituted OC₁-C₆ alkyl; when X is N; and R₂ is H, phenyl or CF₃.
